(19) United States
(12) Patent Application Publication
(10) Pub. No.: US 2003/0106139 A1
(43) Pub. Date: Jun. 12, 2003

(54) Adjustable nose bridge for swimming goggles

(76) Inventor: Bom Kyu Lee, Seoul (KR)

Correspondence Address: Charles N J Ruggiero, Ohlandt Greeley Ruggiero & Perle, 10th Floor, One Landmark Square, Stamford, CT, 06901-2682, US

(21) Appl. No.: 10/258,388

(22) PCT Filed: Apr. 24, 2001
(86) PCT No.: PCT/KR01/00682

(30) Foreign Application Priority Data
Apr. 25, 2000 (KR) 2000-11714

(51) Int. Cl. A61F 9/02

(52) U.S. Cl. 2/426

(57) ABSTRACT

The invention is for swimming goggles 400 with a nose-bridge 420 which can be simply adjusted to lengthen or shorten the space between lenses of the goggles and which is safe for the user even when the swimming goggles are stripped inadvertently. The novel nose-bridge 420 comprises a flexible connecting band 422 having first through-holes 422a towards lateral ends and second throughholes 422b spaced towards the midline from the first through-holes 422a, and supporting members 421 on each of the lenses to connect the two lenses with the flexible connecting band 422. The supporting members 421 on each lens are adapted to have a knob 421a extending towards the midline and each have a supporting hook member 421b having a blunt end 421b′ and a hook end 421b″. This arrangement in combination with the flexible nature, allows for easy adjustment of the nose bridge 420 and avoids injury to the user.

### BACKGROUND OF THE INVENTION

1. Field of the Invention

The present invention relates to swimming goggles, and particularly to swimming goggles with a nose-bridge which can be simply adjusted to adjust the space between lenses of the goggles and which is safe to the user even when the swimming goggles are stripped inadvertently.

2. Description of the Related Art

Conventional swimming goggles have a pair of lens parts disposed within a predetermined distance right and left, and a nose-bridge interconnecting the midline ends of the lenses. Such nose-bridge function to maintain a given distance between interconnecting, midline ends of the pair of lenses, and for adjusting the appropriate space between the pair of lenses.

Referring to FIG. 1 wherein a prior art swimming goggles 100 having a conventional nose-bridge to adjust the space between a pair of lenses are illustrated, nose-bridge 120 connects a pair of lenses 110 by passing both ends of a connecting-band 122, in which widthwise projections 122a, 122b spaced at a predetermined distance are formed on an inner surface thereof, through respective connecting band inserting holes 121 formed on inner ends of the lenses 110. Connecting band inserting holes 121 formed on inner ends of the lenses each have a size that allows the portion of the connecting band, on which projections 122a, 122b are formed, to pass in a biased manner. Thus, when the portion 122c between the projections of the connecting band is positioned in the inserting hole, connecting band 122 is fixed in a state maintaining the given space between lenses 110. If the space between lenses 110 is to be narrowed, a user needs to pass inner projection 122b of the connecting band through connecting band inserting hole 121 formed on the midline ends of lenses 110. This is good enough if connecting band 122 of nose-bridge 120 has to perform only the function of maintaining the space between lenses 110. And connecting band 122 does not need flexibility since projections 122a, 122b formed on inner surface of connecting band 122 should not be easily passed through inserting holes 121. Therefore, the connecting bands of such nose-bridge types are generally required to be made of a rigid-plastic without elasticity.

The ends of connecting band 122 of the swimming goggles are stretched over the top of the bridge of the nose of a user when connecting band 122 is adjusted to narrow the space between lenses 110. As such, the sharp, curved portion of the rigid connecting band 122 may injure the user's skin at the ridge of the nose, especially softened by exposure to water when the swimming goggles 100 are inadvertently (or intentionally) stripped from the user's head.

In addition, as inserting hole 121 has a size that does not allow the portion of connecting band, on which projections 122a, 122b are formed, to pass easily, it is not easy to adjust the space between the lenses.

FIG. 2 depicts another prior art swimming goggles 200 having a nose-bridge 220 with another configuration as illustrated. Nose-bridge 220 has connecting band 222 on which a plurality of through-holes 222a, 222b are spaced at predetermined distances, with pass-holes 221 formed on respective inner (midline) ends of each lens so as to be aligned with any one of said through-holes 222a, 222b, when connecting band 222 is disposed between lenses 210, and fixing pins 223 inserted in any one of through-holes 222a, 222b and pass-holes 221 to affix them thereto, when a respective one of through-holes 222a, 222b and pass-holes 221 are aligned. It is inconvenient and troublesome to adjust the space between lenses 210 through removal of such fixing pins 223, align them with a pass-hole 221 with a desired through-hole, and re-inserting a fixing pin 223. Also such fixing pins 223 are easily misplaced or lost in the process.

FIG. 3 shows another conventional swimming goggles 300 having another prior art example nose-bridge 320. FIG. 3 illustrates nose-bridge 320 as being made up of a flexible connecting band 322 with through-holes 322a/322b respectively, at either ends thereof, and a connecting band support portion 321 formed on inner ends of each lens portions 310 which are linked by the connecting band. Connecting band supporting portions 321 having extending parts 321a extended inwardly from the inner ends of each lens 310, hooks 321b protrude and extend from inner surfaces of extending parts 321a, and protrusions 321c protrude outwardly from outer surfaces of the extending parts 321a. Hooks 321b and protrusions 321c are integrally molded with extending parts 321a of the inner ends of the lenses. Hooks 321b and protrusions 321c of connecting band supporting portion 321 are adapted to be supported on protrusions 321c after hooks 321b are passed into through-holes 322a formed at either end of connecting band 322. Such connecting band 322 has a tendency to inadvertently disengage from connecting band supporting portion 321, and also causes injury to the nose skin of the user weakened by water when the swimming goggles are inadvertently stripped as hooks 321b of connecting band supporting portion 321 confront the wearer's face when such swimming goggles are worn.

The present disclosure describes solutions to the problems in the prior art as identified above.

### SUMMARY OF THE INVENTION

It is an object of the present invention to provide swimming goggles with a connecting band that is easily detachable to facilitate adjustment of the space between the lenses of the swimming goggles, which overcomes the drawbacks of the aforementioned prior art.

It is another object of the present invention to provide swimming goggles having a connecting band with a support member which reliably supports the connecting band to prevent the connecting band from becoming detached from the connecting band-supporting member while in use.

It is another object of the present invention to provide swimming goggles in which the connecting band supporting member is of a conformation and compositional make-up which are safe in preventing injury to a user's face even when the swimming goggles are stripped inadvertently.

Essentially, the swimming goggles or visual protective apparatus for swimming, of the present invention has the requisite pair of lenses connected with a nose-bridge at the midline ends of each lens. As the term is used herein, the “midline” is that portion of the claimed apparatus corresponding to the human midline that runs vertically from head to toe and parallel to the vertical, central surface of the nose. The longitudinal displacement of each lens and nose bridge should be considered perpendicluar to this vertical midline. Thus, if a lens has two longitudinal ends, the “midline end” of a particluar lens is the end towards the anatomical midline of the user and the “lateral end” is the end away from and opposite to the midline end.

In one aspect, the nose-bridge of the present invention is comprised of an independent flexible connecting band and two support members, each on respective lenses. The flexible connecting band can be made of such pliable materials of relatively soft texture as polyurethane.

The connecting band should have at least a first pair of through-holes each sitauted on the two lateral longitudinal ends of the band and at least a second pair of through-holes spaced towards the midline and away from the first through-holes. As already mentioned, a “lateral” disposition refers to a spatial relationshiop away from the vertical midline in a perpendicular, horizanotal direction.

The supporting members are formed at the midline side end of each of said pair of lenses, and are adapted to connect the lenses with the flexible connecting band therebetween. Each supporting member further comprises a knob, a supporting hook member and a bar member connecting the knob to the hook member. Each knob extends from the midline side end of each lens, and each knob has a front surface and a rear surface opposite to the front surface. As used herein, the terms “front” or “outer” or “outside” refers to the surface facing away from the user when the present goggles are worn, and the terms “rear” or “inner” or “inside” refers to the surface facing towards the user's face when the present goggles are worn.

Each supporting member also has a hook member, which has an outer surface and an inner surface opposite to the outer surface, a blunt end and a hook end. The hook end is disposed opposite to the blunt end and extends laterally and a bar member connects the rear surface of the knob to the outer surface of the hook member. The hook end is integrally formed so that it is inclined towards the outer surface and away from the user's nose when the swimming goggles are worn. The remainder of the knob and hook member which are also integral to the lens taper follow the hook end and taper away from the user's nose.

Each of the two lateral longitudinal ends of the flexible connecting band are supported by corresponding supporting members on respective lenses by passing each blunt end of each supporting hook members through the first through-holes of the flexible connecting band, and then pushing each hook end of the supporting hook members through the second through-holes of the flexible connecting band by stretching a portion of the flexible connecting band between each first and second through-holes such that each first through-hole comes to rest around a respective bar member.

### BRIEF DESCRIPTION OF THE DRAWINGS

FIG. 1 is a perspective view of a prior art swimming goggles with a prior art nose-bridge.

FIG. 2 is a perspective view of another prior art swimming goggles with a prior art nose-bridge.

FIG. 3 is a perspective view of a further prior art swimming goggles with a prior art nose-bridge.

FIG. 4 is a perspective view of the swimming goggles in accordance with the present invention showing three variations in size for the inventive connecting band.

FIG. 5 shows one lens with a perspective view of a support member on the lens goggle in accordance with the present invention.

FIG. 6 depicts the lens shown in FIG. 5 along with a partial perspective view of a portion of the inventive connecting band linked and supported to the support member on the lens goggle.

FIG. 7 is a perspective view of one embodiment connecting band of the nose-bridge in accordance with the present invention.

FIG. 8 is a perspective view of another embodiment connecting band of the nose-bridge in accordance with the present invention.

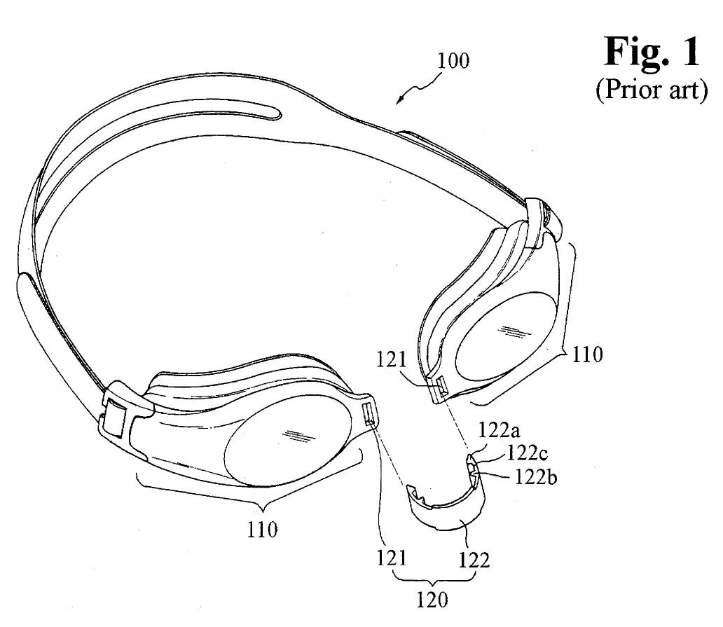

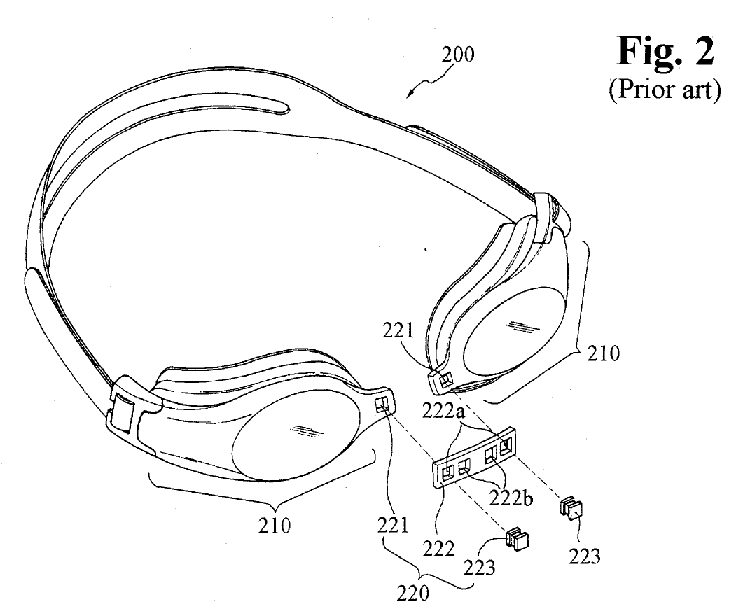

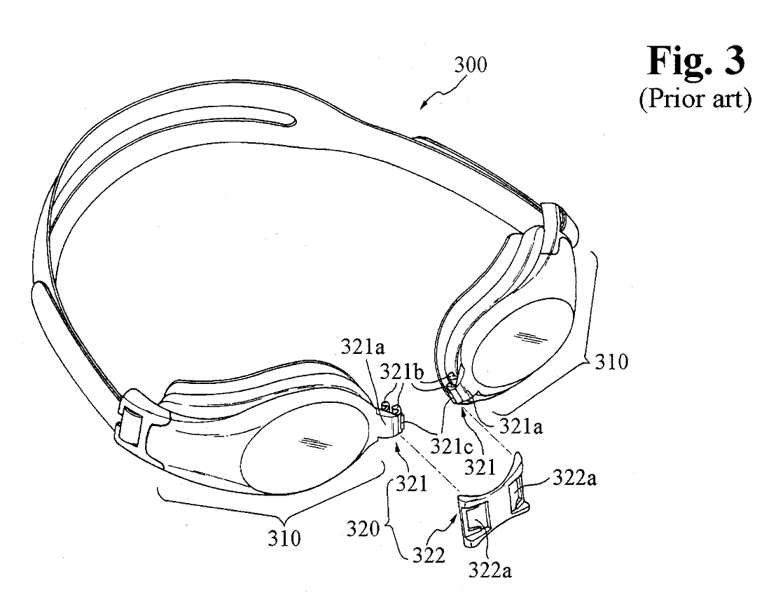

### DETAILED DESCRIPTION OF PREFERRED EMBODIMENTS

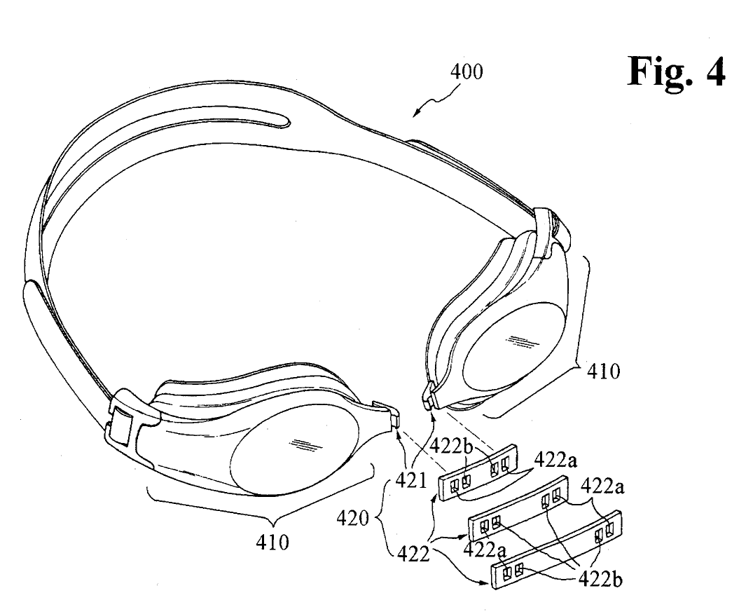

The present invention will now be described in detail with reference to the accompanying drawings. FIG. 4 shows the swimming goggles 400 comprising a pair of lenses 410, and the nose-bridge 420 adapted to connect midline ends of the lenses. As used herein the term “midline end of a lens” means the end of the lens proximate to wearer's nose when the swimming goggles are worn.

Nose-bridge 420 according to the present invention comprises first through-hole 422a formed adjacent either lateral ends of of flexible connecting band 422 which also has second through-hole 422b formed and spaced apart from the first through-hole 422a, and connecting band supporting members 421 formed on midline ends of lenses 410, which are connected by the flexible connecting band 422.

Referring to FIG. 5, supporting member 421 is specifically illustrated without flexible connecting band 422. Each supporting member 421 is formed on a respective midline end of lens 410 and comprises an extending knob 421a, a supporting hook member 421b and a connecting bar member 421c connecting extending knob 421a to supporting hook member 421b. As illustrated, extending knob 421a, supporting hook member 421b and connecting bar member 421c are all integrally formed (such as by molding) with lens 410.

Extending knob 421a extends towards the midline from the midline end of lens 410, and has a rear surface which faces the surface of the user's face when swimming goggles 400 are worn, and a front surface opposite to the rear surface. Supporting hook member 421b has an inside surface which faces the user's face when wearing the swimming goggles, and an outside surface opposite to the inside surface, a blunt end 421b′, and a hook end 421b″. Connecting bar member 421c connects the outside surface of hook member 421b with the rear surface of extending knob 421a.

The hook end 421″ is integrally formed so that it is inclined towards the outer surface and away from the user's nose when the swimming goggles are worn. The remainder of the hook member 421b and the knob 421a, which are all also integral with the lens, and are adapted to taper away from the user's nose as illustrated in FIG. 5. Thus, extending knob 421a inclines away from the center line of the nose of the user in order to provide more space away from the user's face. In this way, there is little danger of connecting with the nose of a person with a high nose ridge or otherwise. Also, there is little probability that the nose-bridge will contact the user's face if the swimming goggles fall off inadvertently. Therefore, there is little risk that the user will suffer injury.

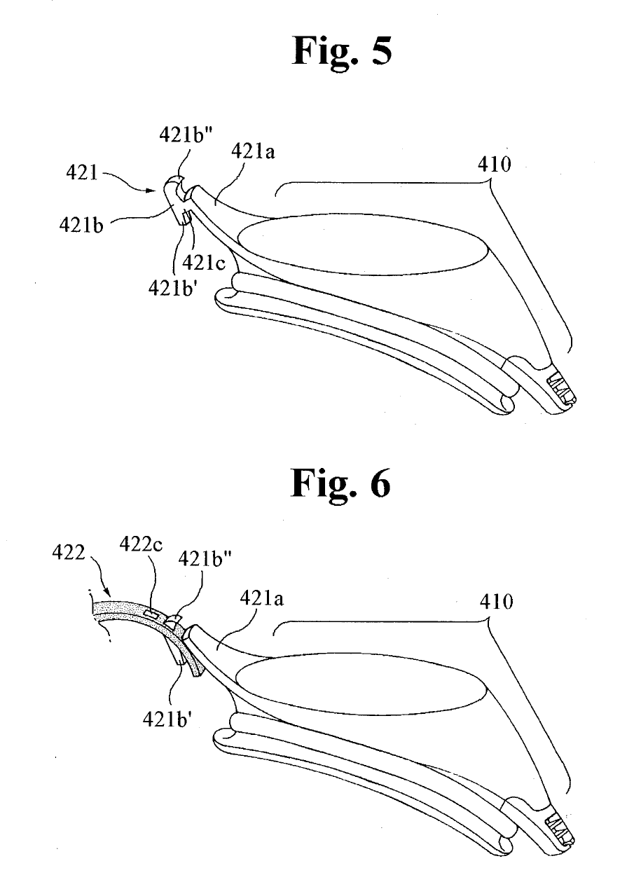

FIG. 6 shows the positioning of the flexible connecting band 422 on supporting hook member 421b and connecting bar member 421c which are integral with extending knob 421a of the supporting member 421. The blunt end 421b′ of supporting hook member 421b are adapted to be passed through first through hole 422a of flexible connecting band 422 (cross reference FIG. 6 with FIG. 4). The hook end 421b″ (FIG. 6) are adapted to be inserted into second through hole 422b of flexible connecting band 422 (FIG. 4) by stretching flexible connecting band 422 so that when the ends of flexible connecting band 422 are supported by corresponding supporting means 421, first through hole 422a is positioned around connecting bar member 421c.

Through use of components which make up the nose bridge 420 of the present invention, connecting band 422 can be easily installed onto supporting member 421. Once installed, connecting band 422 is not easily dislodged from the supporting member as the hook end 421b″ holds it in place with second through hole 422b held firmly around bar member 421c.

The elements of the supporting member 421 are designed such that compact integral manufacture in relatively smaller diemensions compared to the prior art, are possible and thus occupy only a small space in the overall swimming goggles configuration. Thus, the overall design of the swimming goggles is varied to provide a safe structure that avoids injury to the user's face when the swimming goggles is inadvertently displaced or intentionally removed.

In order for easy installation of connecting band 422 onto swimming goggles 400, the connecting band should have some resilience. However, to conduct the function of adjusting the space between the lenses, the resilience should not be excessively high. Thus, the connecting band according to present invention is preferably made of polyurethane. Other materials which can provide such requirements as ease of installation, but not excessive resilience for adjustment, and provide safe texture, can also be substituted by one skilled in the art. All such substitutions, which will become apparent after having the benefit of this disclosure, are considered to be within the scope of this patent.

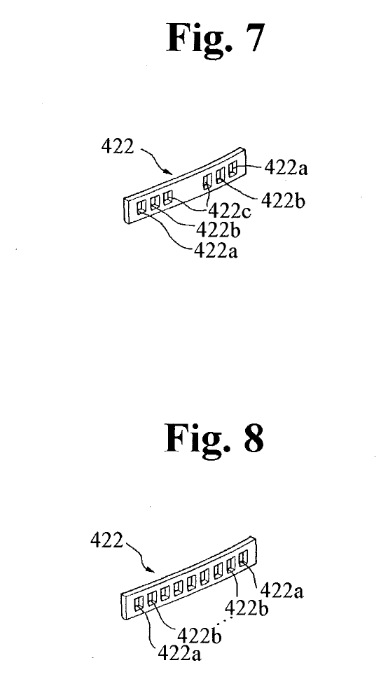

The illustrative embodiment has been shown with four through-holes in connecting band 422 (FIG. 4). First and second through holes 422a, 422b are formed towards the lateral longitudinal ends of the connecting band in the illustrated embodiment. FIG. 4 shows three different lengths wherein the space between the second through holes 422b can be varied. It will become apparent after having the benefit of this disclosure that a plurality of through holes beyond four are possible. For example, as illustrated in FIG. 7, third through holes 422c can be spaced toward the midline from the second through-holes 422b of the flexible connecting band 422. Thus, the space between lenses 410 can be narrowed by adjusting the second through-holes 422b and third through-holes 422c between the hook and connection bar member 421c of hook end 421b″ on the outside surface of supporting hook member 421b, when the end of flexible connecting band 422 is supported on respective supporting member 421 by stretching the flexible connecting band 422 and passing hook end 421b″ of supporting hook member 421b through third through-holes 422c of flexible connecting band 422, after passing blunt end 421b′ through second through-holes 422b. In addition, as illustrated in FIG. 8, a connecting band on which a plurality of through holes is formed in sequence throughout its entire length can also be used. In such a case, the number of through holes formed on the connecting band should be more than five.

The length of the flexible connecting band 422 which connects the gap between lenses 410 can be properly adjusted by preparing a set of flexible connecting band 422 whose lengths are varied such as long, medium or short, and marketing a choice for fit depending on the variable space between the user's eyes.

Illustrative embodiments of the invention have been described, and it will be understood that many changes and modifications can be made thereto without departing from the spirit or scope of the invention.

What is claimed is:

1. A visual protective apparatus for swimming which has a pair of lenses and a nose-bridge interconnecting the midline ends of said lenses, said apparatus characterized with the nose-bridge comprising:
a) a flexible connecting band having first through-holes on lateral longitudinal ends of said band and second through-holes spaced towards the midline from said first through-holes; and
b) supporting members formed at the midline side end of each of said pair of lenses, said supporting members adapted to connect said lenses with said flexible connecting band therebetween;
wherein each said supporting member further comprises a knob extending from the midline side ends of each of said pair of lenses, said knob having a front surface and a rear surface opposite to the front surface; a supporting hook member having an outer surface and an inner surface opposite to the outer surface, a blunt end and a hook end, said hook end disposed opposite to the blunt end and extending laterally; and a bar member connecting the rear surface of said knob to the outer surface of said hook member; and
wherein each said lateral longitudinal ends of said flexible connecting band is supported by corresponding supporting members on respective lenses by passing each said blunt ends of each supporting hook members through said first through-holes of said flexible connecting band, and then pushing each said hook ends of the supporting hook members through said second through-holes of said flexible connecting band by stretching a portion of said flexible connecting band between each said first through-hole and each said second through-hole such that each said first through-hole comes to rest around respective said bar members.

2. Swimming goggles made up of a first and a second lenses and a nose-bridge connecting midline side ends of each said first and second lenses, and characterized with said nose-bridge comprising:
a) a flexible connecting band having at least five through-holes formed through its entire length; and
b) a first supporting member formed at a midline end of said first lens and a second supporting member formed at a midline end of said second lens, said flexible connecting band linking together said lenses such that each said lens are connected to said flexible connecting band at each respective midline end of each said lens;
wherein each said supporting member has a knob extending towards the midline from the midline side.end of each lens, said knob having a front surface and a rear surface opposite to the front surface; a supporting hook member having an inside surface and an outside surface opposite to the inside surface, a blunt end and a hook end opposite said blunt end; and a bar member connecting said knob to said hook member; and
wherein each said lateral longitudinal ends of said flexible connecting band is supported by respective said first supporting member of said first lens and said second supporting member of said second lens, by passing each said blunt end of each supporting hook member through a laterally situated through-hole of said flexible connecting band and then passing said hook end of the supporting hook member through another through-hole situated midline from said laterally situated through-hole by stretching said flexible connecting band between each said laterally situated through-hole and each said through-hole hole situated midline from said laterally situated through-hole, such that each said laterally situated through-hole comes to rest around each respective bar member.

3. A visual protective apparatus for swimming as in claim 1, wherein said flexible connecting band is made of polyurethane.
